(19) United States
(12) Patent Application Publication
(10) Pub. No.: US 2024/0180634 A1
(43) Pub. Date: Jun. 6, 2024

(54) SURGICAL NAVIGATION SYSTEMS AND METHODS INCLUDING MATCHING OF MODEL TO ANATOMY WITHIN BOUNDARIES

(71) Applicant: PolarisAR, Inc., Miami, FL (US)

(72) Inventor: Paul W. Mikus, Miami, FL (US)

(73) Assignee: PolarisAR, Inc., Miami, FL (US)

(21) Appl. No.: 18/556,078

(22) PCT Filed: Apr. 20, 2022
(86) PCT No.: PCT/US2022/025647

§ 371 Date: Oct. 18, 2023

Related U.S. Application Data
(60) Provisional application No. 63/177,708, filed on Apr. 21, 2021.

(51) Int. Cl. A61B 34/20; A61B 34/10; A61B 90/00; A61B 90/50

(52) U.S. Cl. CPC A61B 34/20; A61B 34/10; A61B 90/36; A61B 90/50; A61B 2034/105; A61B 2034/2055; A61B 2090/365; A61B 2090/367; A61B 2090/502

(57) ABSTRACT

Surgical navigation systems and methods may match a model to an anatomy within sections defined by boundaries. Once the sections are matched, the sections as well as other portions of (e.g., the entire) model may be registered to an intraoperative scene. The model may be a three-dimensional model generated from a plurality of two-dimensional images of a portion of a body of a patient. The surgical navigation systems and methods may reduce and/or obviate a need for a marker, such as a fiducial, a tracker, an optical code, a tag, or a combination thereof during a medical procedure.

### CROSS-REFERENCE TO RELATED APPLICATIONS

This application claims the benefit under 35 U.S.C. § 119(e) of the earlier filing date of U.S. Provisional Application No. 63/177,708 filed Apr. 21, 2021, the entire contents of which are hereby incorporated by reference in their entirety for any purpose.

### BACKGROUND

Medical professionals may utilize surgical navigation systems to provide a surgeon(s) with assistance in identifying precise locations for surgical applications of devices, resection planes, targeted therapies, instrument or implant placement, or other complex procedural approaches. Some benefits of the surgical navigation systems may include allowing for real time (or near real time) information that the surgeon may utilize during a surgical intervention. Current surgical navigation systems may rely on a need to employ some type of marker at or near an anatomical treatment site, often as part of an overall scheme to determine the precise location.

The markers, often in the form of fiducials, trackers, optical codes, tags, and so forth, may require a precise setup in order to be effective. Unfortunately, a considerable setup time and a considerable complexity may be a deterrent(s) for the medical professionals to use the current surgical navigation systems. In addition, the use of markers at the anatomical sites, and instruments used in a procedure may need to be referenced continually in order to maintain a reference location status. An interference(s) with a line of sight between cameras used to capture images of the markers may disrupt the referencing, and ultimately, a navigation of a surgical process as a whole.

### SUMMARY

Example surgical navigation methods are disclosed herein. In an embodiment of the disclosure, an example surgical navigation method includes receiving a plurality of two-dimensional images of a portion of a body of a patient. From the two-dimensional images, the surgical navigation method generates a three-dimensional reconstructed model of the portion of the body. The surgical navigation method includes generating a model boundary in the three-dimensional reconstructed model based on a section of interest. The surgical navigation method includes receiving an intraoperative image of the at least a portion of the body. The surgical navigation method includes generating a live anatomy boundary based on the intraoperative image. The surgical navigation method includes matching digital samples from within the model boundary with digital samples from within the live anatomy boundary to register the three-dimensional reconstructed model with the at least a portion of the body.

Additionally, or alternatively, a non-transitory computer-readable storage medium includes instructions that, when executed by a processor, configures the processor to perform said surgical navigation method.

Additionally, or alternatively, said matching of the digital samples from within the model boundary with the digital samples from within the live anatomy boundary obviates a need for a fiducial, a tracker, an optical code, a tag, or a combination thereof.

Additionally, or alternatively, said receiving of the intraoperative image comprises obtaining the intraoperative image using an augmented reality device during a medical procedure.

Additionally, or alternatively, the model boundary aids a medical provider during a pretreatment process, a preoperative process, an intraoperative process, a postoperative process, or a combination thereof of a medical procedure.

Additionally, or alternatively, the matching of the digital samples from within the model boundary with the digital samples from within the live anatomy boundary is performed by utilizing: an iterative closest point algorithm; a machine-learned model for matching one or more patterns of the digital samples from within the model boundary to one or more patterns of the digital samples from within the live anatomy boundary; or a combination thereof.

Additionally, or alternatively, the model boundary comprises a two-dimensional area, the two-dimensional area being defined by one or more of geometric shapes, and the one or more geometric shapes comprising a line, a regular polygon, an irregular polygon, a circle, a partial circle, an ellipse, a parabola, a hyperbola, a logarithmic-function curve, an exponential-function curve, a convex curve, a polynomial-function curve, or a combination thereof.

Additionally, or alternatively, the model boundary comprises a three-dimensional volumetric region, the three-dimensional volumetric region being defined by a cuboid, a polyhedron, a cylinder, a sphere, a cone, a pyramid, a prism, a torus, or a combination thereof.

Additionally, or alternatively, the model boundary comprises a surface with a relief.

Additionally, or alternatively, the model boundary comprises a shape, the shape being drawn by a medical professional.

Additionally, or alternatively, the live anatomy boundary comprises approximately a same size, shape, form, location on the portion of the body, or a combination thereof as the model boundary.

Example systems for aiding a medical provider during a medical procedure are disclosed herein. In an embodiment of the disclosure, an example system includes an augmented reality headset, a processor, and a non-transitory computer-readable storage medium including instructions. The instructions of the con-transitory computer-readable storage medium, when executed by the processor, cause the system to: receive an indication of a live anatomy boundary for an intraoperative scene: display, using the augmented reality headset, the live anatomy boundary overlaid on the intraoperative scene: receive an indication of an alignment of the live anatomy boundary with a section of interest of at least a portion of a body: and match a section of a pretreatment image defined by a pretreatment boundary with a section of an intraoperative image associated with the live anatomy boundary to register the pretreatment image with the intraoperative scene.

Additionally, or alternatively, the instructions, when executed by the processor, further cause the system to match digital samples from within the live anatomy boundary with digital samples from within a model boundary associated with the pretreatment image of the portion of the body.

Additionally, or alternatively, the model boundary is based on a three-dimensional reconstructed model of the portion of the body.

Additionally, or alternatively, the matching of the digital samples aid the system to register the three-dimensional reconstructed model with the at least the portion of the body.

Additionally, or alternatively, the system comprises a markerless surgical navigation system.

Additionally, or alternatively, the instructions, when executed by the at least one processor, further cause the system to establish communication between the augmented reality headset and one or more of a pretreatment computing device, a surgical navigation computing device, and a registration computing device.

Additionally, or alternatively, the live anatomy boundary comprises a virtual object.

Additionally, or alternatively, the instructions, when executed by the processor, further cause the system to: generate a model boundary from a first input of a first medical professional during a pretreatment process of the medical procedure, the first input comprises the first medical professional utilizing the pretreatment computing device: and generate the live anatomy boundary from a second input of a second medical professional during an intraoperative process of the medical procedure, the second input comprises the second medical professional utilizing the augmented reality device to: indicate the live anatomy boundary of the intraoperative image: indicate the alignment of the live anatomy boundary with the section of interest of the at least a portion of the body: or a combination thereof.

Additionally, or alternatively, the instructions further cause the system to provide guidance for a surgical procedure based on a registration of the pretreatment image with the intraoperative scene.

### BRIEF DESCRIPTION OF THE DRAWINGS

FIG. 1 illustrates an example environment of a markerless surgical navigation system in accordance with examples described herein.

FIG. 2A illustrates an example diagram of a pretreatment computing device in accordance with examples described herein.

FIG. 2B illustrates an example diagram of an augmented reality device in accordance with examples described herein.

FIG. 2C illustrates an example diagram of a surgical navigation computing device in accordance with examples described herein.

FIG. 2D illustrates an example diagram of a registration computing device in accordance with examples described herein.

FIG. 3A illustrates an example two-dimensional image of a portion of a body in accordance with examples described herein.

FIG. 3B illustrates an example three-dimensional reconstructed model (3D reconstructed model) of the portion of the body in accordance with examples described herein.

FIG. 4 illustrates an example method for registering the 3D reconstructed model of a portion of a body with an the actual (live) portion of the body of the patient during, the patient being during and/or in an intraoperative process of a medical procedure, in accordance with examples described herein.

FIG. 5A illustrates an example model boundary of the 3D reconstructed model generated during a pretreatment process of the medical procedure in accordance with examples described herein.

FIG. 5B illustrates an example live anatomy boundary of an intraoperative image generated during an intraoperative process of the medical procedure in accordance with examples described herein.

### DETAILED DESCRIPTION

Examples described herein include surgical navigation systems that may operate to register pre-operative or other anatomical models with anatomical views from intraoperative imaging without a need for the use of fiducials or other markers. While not all examples may have all or any advantages described or solve all or any disadvantages of systems utilizing markers, it is to be appreciated that the setup time and complexity of systems utilizing markers may be a deterrent. The simplicity and ease of use of markerless systems described herein may be advantageous. In some examples, markerless registration may be used in systems that also employ markers or other fiducials to verify registration and/or perform other surgical navigation tasks. Examples of surgical navigation systems described herein, however, may maintain the precision of existing marker-based surgical navigation systems using markerless registration. Disclosed herein may be examples of markerless surgical navigation system(s) and method(s) that may be simple to setup, can be configured for a multitude of surgical applications, and can be deployed with technologies, such as an augmented reality and/or robotics technology(ies) to improve a usability and a precision during a medical procedure.

In one aspect, a surgical navigation method includes receiving a plurality of two-dimensional images of a portion of a body of a patient. From the two-dimensional images, the surgical navigation method may generate a three-dimensional reconstructed model of the portion of the body. The surgical navigation method includes generating a model boundary in the three-dimensional reconstructed model based on a section of interest. At a different time, for example, at a later time, the surgical navigation method includes receiving an intraoperative image of the at least a portion of the body. The surgical navigation method may include generating a live anatomy boundary based on the intraoperative image. The live anatomy boundary may be based on the same section of interest as the model boundary. The surgical navigation method may include matching digital samples from within the model boundary with digital samples from within the live anatomy boundary. By so doing, the surgical navigation method can register the three-dimensional reconstructed model with the at least a portion of the body.

In one aspect, a system, such as a markerless surgical navigation system or a surgical navigation system, may aid a medical provider (e.g., a surgeon) that may be utilizing an augmented reality headset during a medical procedure. The system includes a processor and a non-transitory computer-readable storage medium that may store instructions, and the system may utilize the processor to execute the instructions to perform various tasks. For example, the system may display an intraoperative image, using the augmented reality headset, of at least a portion of a body of a patient. The system may also receive an indication, for example, from the medical provider, of a live anatomy boundary of the intraoperative image. The system may display, using the augmented reality headset and/or another computing system, the live anatomy boundary and the intraoperative image. The system may receive an indication, for example, from the medical provider, of an alignment of the live anatomy boundary with a section of interest of the at least a portion of the body. The system may also display, on the augmented reality headset, the live anatomy boundary aligned with the section of interest. The system may match digital samples from within the model boundary with digital samples from within the live anatomy boundary to register the three-dimensional reconstructed model with the at least a portion of the body. By so doing, the system (e.g., the markerless surgical navigation system) may reduce and/or obviate a need for a marker, such as a fiducial, a tracker, an optical code, a tag, or a combination thereof.

In aspects, a system, an apparatus, an application software, portions of the application software, an algorithm, a model, and/or a combination thereof may include performing the surgical navigation method mentioned above. For example, a system may include and/or utilize one or more computing devices and/or an augmented reality device to perform the surgical navigation methods and/or registration methods described herein. As another example, at least one non-transitory computer-readable storage medium may include instructions that, when executed by at least one processor, may cause one or more computing systems and/or augmented reality headsets to perform surgical navigation methods and/or registration methods described herein.

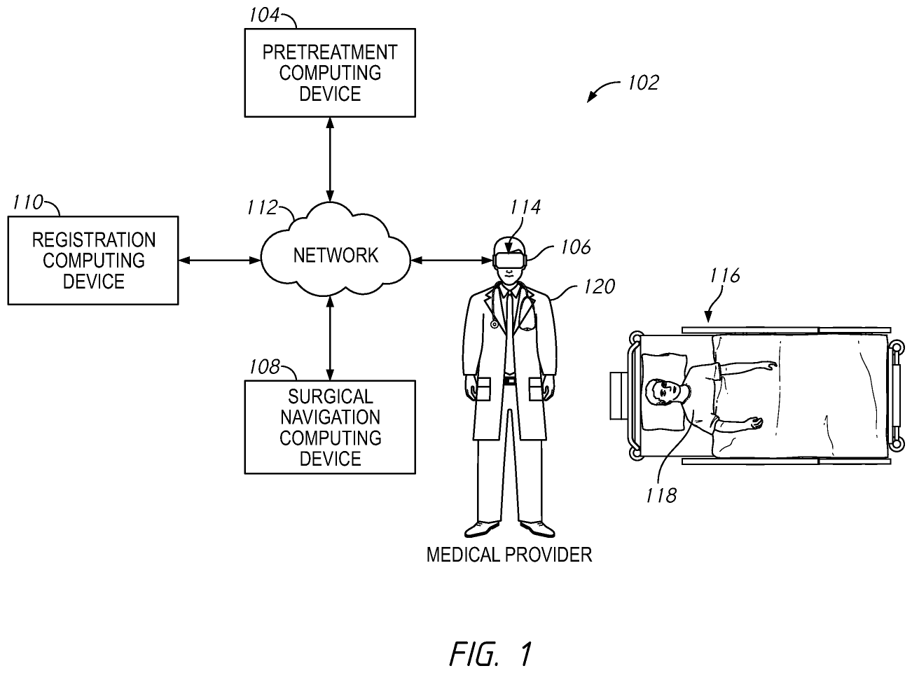

FIG. 1 illustrates an example a system 102 (e.g., markerless surgical navigation system 102 and/or surgical navigation system 102). In some examples, the surgical navigation system 102 may include a pretreatment computing device 104, an augmented reality device 106, a surgical navigation computing device 108, and a registration computing device 110.

FIG. 1 illustrates the augmented reality device 106, the surgical navigation computing device 108, and the registration computing device 110 as distinct (or separate) computing devices. Nevertheless, the augmented reality device 106, the surgical navigation computing device 108, and/or the registration computing device 110 may be combined and/or integrated in numerous ways. For example, the surgical navigation computing device 108 and the registration computing device 110 may be combined and/or integrated into a first computing device, and the augmented reality device 106 may be a second computing device. As another example, the augmented reality device 106 and the surgical navigation computing device 108 may be combined and/or integrated into a first computing device, and the registration computing device 110 may be a second computing device. As another example, the augmented reality device 106, the surgical navigation computing device 108, and the registration computing device 110 may be integrated and/or combined in a single computing device. As yet another example, one or more of the surgical navigation computing device 108, and/or the registration computing device 110 may be optional, for example, when used in conjunction with the augmented reality device 106.

In some embodiments, the various devices of the surgical navigation system 102 may communicate with each other directly and/or via a network 112. The network 112 may facilitate communication between the pretreatment computing device 104 the augmented reality device 106, the surgical navigation computing device 108, the registration computing device 110, a satellite(s) (not and/or illustrated), a base station(s) (not illustrated). Communication(s) in the surgical navigation system 102 may be performed using various protocols and/or standards. Examples of such protocols and standards, include: a 3rd Generation Partnership Project (3GPP) Long-Term Evolution (LTE) standard, such as a 4th Generation (4G) or a 5th Generation (5G) cellular standard: an Institute of Electrical and Electronics (IEEE) 802.11 standard, such as IEEE 802.11g, ac, ax, ad, aj, or ay (e.g., Wi-Fi 6®; or WiGig®); an IEEE 802.16 standard (e.g., WiMAX®); a Bluetooth Classic®; standard; a Bluetooth Low Energy® or BLE® standard; an IEEE 802.15.4 standard (e.g., Thread® or ZigBee®); other protocols and/or standards that may be established and/or maintained by various governmental, industry, and/or academia consortiums, organizations, and/or agencies: and so forth. Therefore, the network 112 may be a cellular network, the Internet, a wide area network (WAN), a local area network (LAN), a wireless LAN (WLAN), a wireless personal-area-network (WPAN), a mesh network, a wireless wide area network (WWAN), a peer-to-peer (P2P) network, and/or a Global Navigation Satellite System (GNSS) (e.g., Global Positioning System (GPS), Galileo, Quasi-Zenith Satellite System (QZSS), BeiDou, GLObal NAvigation Satellite System (GLONASS), Indian Regional Navigation Satellite System (IRNSS), and so forth).

In addition to, or alternatively of, the communications illustrated in FIG. 1, the surgical navigation system 102 may facilitate other unidirectional, bidirectional, wired, wireless, direct, and/or indirect communications utilizing one or more communication protocols and/or standards. Therefore, FIG. 1 does not necessarily illustrate all communication signals.

In some embodiments, the surgical navigation system 102 may display a virtual environment 114 via and/or using (e.g., on) the augmented reality device 106. The virtual environment 114 may be a wholly virtual environment and/or may include one or more virtual objects. Alternatively, or additionally, the virtual environment 114 (e.g., one or more virtual objects) may be combined with a view of a real environment 116 to generate an augmented (or a mixed) reality environment of, for example, a portion of a body of a patient 118. The augmented reality environment of the portion of the body of the patient 118 may aid a medical provider 120 during a medical procedure. Generally, the medical procedure may include a pretreatment process, a preoperative process, an intraoperative process, a postoperative process, or a combination thereof. In some embodiments, this disclosure may focus on the preoperative process and the intraoperative process of the medical procedure.

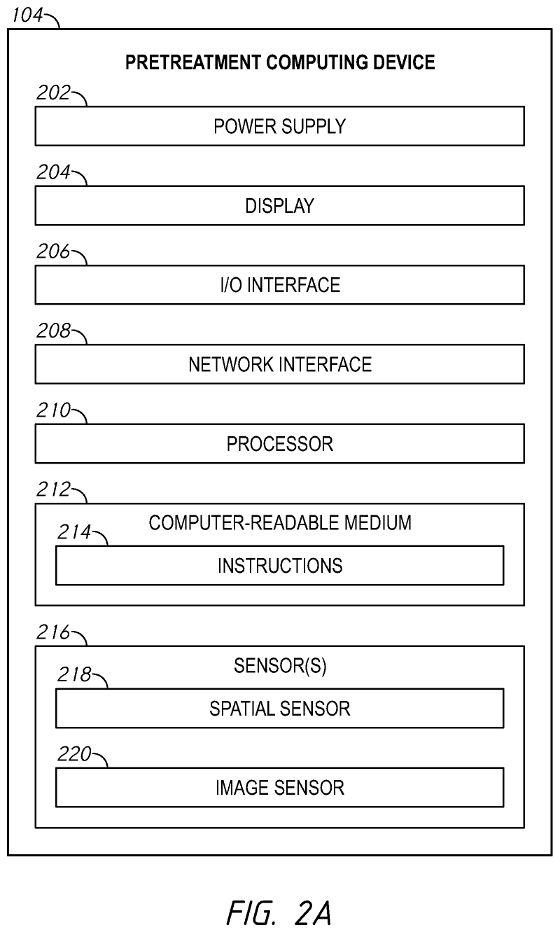

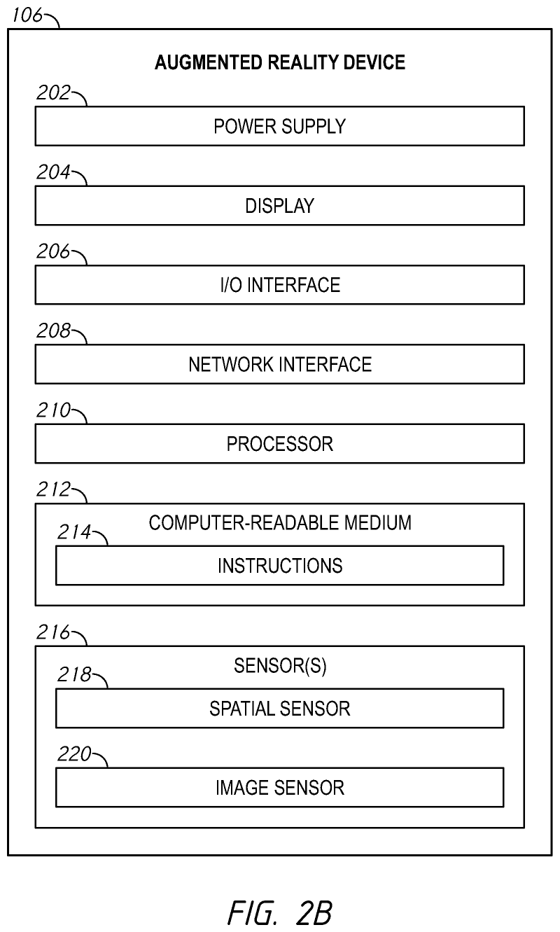

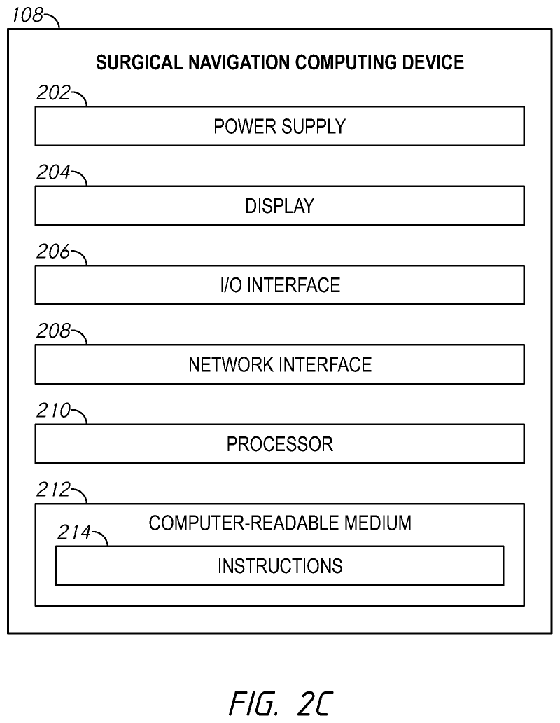

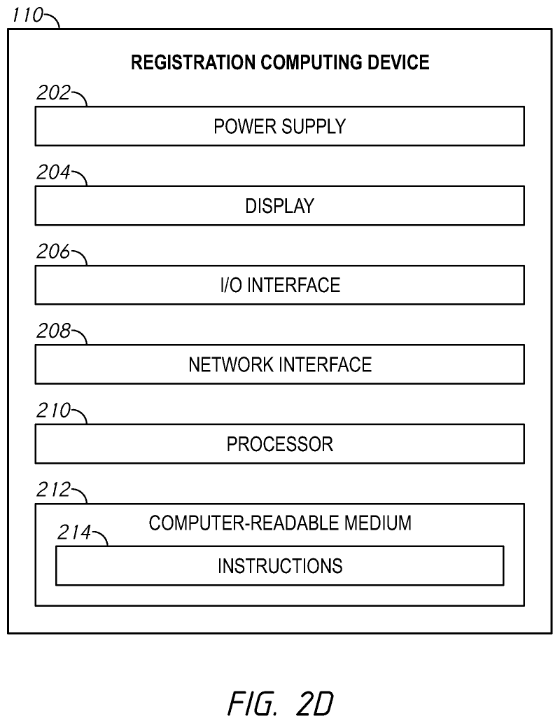

FIGS. 2A, 2B, 2C, and 2D illustrate example diagrams of the pretreatment computing device 104, the augmented reality device 106, the surgical navigation computing device 108, and the registration computing device 110, respectively. In one embodiment, for example, as is illustrated in FIGS. 2A, 2B, 2C, and 2D each of the pretreatment computing device 104, the augmented reality device 106, the surgical navigation computing device 108, and registration computing device 110 may include a power supply 202, a display 204, an input/output interface 206 (I/O interface 206), a network interface 208, an application processor (processor 210), and a computer-readable medium 212 that includes computer-executable instructions 214 (instructions 214) (e.g., code, pseudocode, instructions which may implement one or more algorithm(s), such as an iterative closest point algorithm, instructions which may implement a machine-learned model, or other instructions). Furthermore, the pretreatment computing device 104 of FIG. 2A and the augmented reality device 106 of FIG. 2B may also include and/or utilize sensor(s) 216, for example, a spatial sensor 218, an image sensor 220, and/or other sensors that may not be explicitly illustrated in FIGS. 2A and 2B. Some of the components illustrated in FIGS. 2A, 2B, 2C, 2D, however, may be optional.

For brevity, the power supply 202, the display 204, the I/O interface 206, the network interface 208, the processor 210, the computer-readable medium 212, and the instructions 214 of FIGS. 2A, 2B, 2C, 2D share the same number. Similarly, still for the sake of brevity, the sensors 216, the spatial sensor 218, and the image sensor 220 of FIG. 2A and FIG. 2B share the same number. Nevertheless, these components may be the same, equivalent, or different for the pretreatment computing device 104 of FIG. 2A, the augmented reality device 106 of FIG. 2B, the surgical navigation computing device 108 of FIG. 2C, and registration computing device 110 of FIG. 2D.

In some embodiments, the power supply 202 (of any of the FIGS. 2A, 2B, 2C, and/or 2D) may provide power to various components within the pretreatment computing device 104 of FIG. 2A, the augmented reality device 106 of FIG. 2B, the surgical navigation computing device 108 of FIG. 2C, and/or the registration computing device 110 of FIG. 2D. In some embodiments, one or more devices of the surgical navigation system 102 of FIG. 1 may share the power supply 202. Also, the power supply 202 may include one or more rechargeable, disposable, or hardwire sources, for example, a battery(ies), a power cord(s), an alternating current (AC) to direct current (DC) inverter (AC-to-DC inverter), a DC-to-DC converter, and/or the like. Additionally, the power supply 202 may include one or more types of connectors or components that provide different types of power (e.g., AC power, DC power) to any of the devices of the surgical navigation system 102 of FIG. 1, such as the pretreatment computing device 104, the augmented reality device 106, the surgical navigation computing device 108, the registration computing device 110, the network 112, and/or the like. The power supply 202 may also include a connector (e.g., a universal serial bus) that provides power to any device or batteries within the device. Additionally, or alternatively, the connector of the power supply 202 may also transmits data to and from the various devices of the surgical navigation system 102 of FIG. 1.

In some embodiments, the display 204 may be optional in one or more of the devices of the surgical navigation system 102 of FIG. 1, the pretreatment computing device 104 of FIG. 2A, the augmented reality device 106 of FIG. 2B, the surgical navigation computing device 108 of FIG. 2C, and/or the registration computing device 110 of FIG. 2D. If any of the aforementioned devices includes and/or utilizes a display, the display 204 may display visual information, such as an image(s), a video(s), a graphical user interface (GUI), notifications, and so forth to a user (e.g., the medical provider 120 of FIG. 1). The display 204 may utilize a variety of display technologies, such as a liquid-crystal display (LCD) technology, a light-emitting diode (LED) backlit LCD technology, a thin-film transistor (TFT) LCD technology, an LED display technology, an organic LED (OLED) display technology, an active-matrix OLED (AMOLED) display technology, a super AMOLED display technology, and so forth. Furthermore, in the augmented reality device 106, the display 204 may also include a transparent or semi-transparent element, such as a lens or waveguide, that allows the medical provider 120 to simultaneously see the real environment 116 and information or objects projected or displayed on the transparent or semi-transparent element, such as virtual objects in the virtual environment 114. The type and number of displays 204 may vary with the type of a device (e.g., a pretreatment computing device 104, an augmented reality device 106, a surgical navigation computing device 108, a registration computing device 110). In some examples, the augmented reality device 106 may be implemented using a HoloLens R: headset.

Furthermore, for one or more of the pretreatment computing device 104, the augmented reality device 106, the surgical navigation computing device 108, and the registration computing device 110, the display 204 may be a touchscreen display that may utilize any type of touchscreen technology, such as a resistive touchscreen, a surface capacitive touchscreen, a projected capacitive touchscreen, a surface acoustic wave (SAW) touchscreen, an infrared (IR) touchscreen, and so forth. In such a case, the touchscreen (e.g., the display 204 being a touchscreen display) may allow the medical provider 120 to interact with any of the devices of the surgical navigation system 102 of FIG. 1. For example, using a GUI displayed on the display 204, the medical provider 120 may make a selection of and/or within a two-dimensional image (2D image) of a portion of a body of a patient (e.g., the patient 118 of FIG. 1), a three-dimensional reconstructed model (3D reconstructed model) of the portion of the body, a model boundary in the 3D reconstructed model based on a section of interest, an intraoperative image of the portion of the body, a live anatomy boundary based on the intraoperative image, and/or so forth, as described herein.

In some embodiments, the I/O interface 206 of any of the pretreatment computing device 104, the augmented reality device 106, the surgical navigation computing device 108, and/or the surgical navigation computing device 108 may allow these devices to receive an input(s) from a user (e.g., the medical provider 120) and provide an output(s) to the same user (e.g., the same medical provider 120) and/or another user (e.g., a second medical provider, a second user). In some embodiments, the I/O interface 206 may include, be integrated with, and/or may operate in concert and/or in situ with another component of any of the pretreatment computing device 104, the augmented reality device 106, the surgical navigation computing device 108, the registration computing device 110, and the network 112, and/or so forth. For example, the I/O interface 206 may include a touchscreen (e.g., a resistive touchscreen, a surface capacitive touchscreen, a projected capacitive touchscreen, a SAW touchscreen, an IR touchscreen), a keyboard, a mouse, a stylus, an eye tracker, a gesture tracker (e.g., a camera-aided gesture tracker, an accelerometer-aided gesture tracker, a gyroscope-aided gesture tracker, a radar-aided gesture tracker, and/or so forth), and/or the like. The type(s) of the device(s) that may interact using the I/O interface 206 may be varied by, for example, design, preference, technology, function, and/or other factors.

In some embodiments, the network interface 208 illustrated in any of the FIGS. 2A, 2B, 2C, and/or 2D may enable any of the pretreatment computing device 104, the augmented reality device 106, the surgical navigation computing device 108, and the registration computing device 110 to receive and/or transmit data directly to any of the network interfaces 208 of said devices. Alternatively, or additionally, the devices illustrated in FIGS. 1, 2A, 2B, 2C, and 2D may utilize their respective network interfaces (e.g., the network interface 208) to communicate with each other indirectly by, for example, using the network 112 of FIG. 1.

In some embodiments, the network interface 208 illustrated in any of the FIGS. 2A, 2B, 2C, and/or 2D may include and/or utilize an application programming interface (API) that may interface and/or translate requests across the network 112 of FIG. 1 to the pretreatment computing device 104, the augmented reality device 106, the surgical navigation computing device 108, and/or the registration computing device 110. It is to be understood, that the network interface 208 may support a wired and/or a wireless communication using any of the aforementioned communication protocols and/or standards.

In some embodiments, the processor 210 illustrated in any of the FIGS. 2A, 2B, 2C, and 2D may be substantially any electronic device that may be capable of processing, receiving, and/or transmitting the instructions 214 that may be included in, permanently or temporarily saved on, and/or accessed by the computer-readable medium 212. In aspects, the processor 210 may be implemented using one or more processors (e.g., a central processing unit (CPU), a graphic processing unit (GPU)), and/or other circuitry, where the other circuitry may include as at least one or more of a application specific integrated circuit (ASIC), a field programmable gate array (ASIC), a microprocessor, a microcomputer, and/or the like. Furthermore, the processor 210 may be configured to execute the instructions 214 in parallel, locally, and/or across the network 112 of FIG. 1, for example, by using cloud and/or server computing resources.

In some embodiments, the computer-readable medium 212 illustrated in any of FIGS. 2A, 2B, 2C, and 2D may be and/or include any suitable data storage media, such as volatile memory and/or non-volatile memory. Examples of volatile memory may include a random-access memory (RAM), such as a static RAM (SRAM), a dynamic RAM (DRAM), or a combination thereof. Examples of non-volatile memory may include a read-only memory (ROM), a flash memory (e.g., NAND flash memory, NOR flash memory), a magnetic storage medium, an optical medium, a ferroelectric RAM (FeRAM), a resistive RAM (RRAM), and so forth. Moreover, the computer-readable medium 212 does not include transitory propagating signals or carrier waves.

The instructions 214 that may be included in, permanently or temporarily saved on, and/or accessed by the computer-readable medium 212 of any of the FIGS. 2A, 2B, 2C, and 2D may include code, pseudo-code, algorithms, models, and/or so forth and are executable by the processor 210. In addition to the instructions 214, the computer-readable medium 212 of any of the FIGS. 2A, 2B, 2C, and 2D may also include other data, such as audio files, video files, digital images, 2D images, medical information regarding the patient 118 of FIG. 1, a 3D reconstructed model, and/or other data that may aid the medical provider 120 of FIG. 1 during a pretreatment process, a preoperative process, an intraoperative process, and/or a postoperative process of the medical procedure. It is to be understood that the instructions 214 may be different for the pretreatment computing device 104 of FIG. 2A, the augmented reality device 106 of FIG. 2B, the surgical navigation computing device 108 of FIG. 2C, and/or the registration computing device 110 of FIG. 2D.

In some embodiments, the pretreatment computing device 104 of FIG. 2A and/or the augmented reality device 106 of FIG. 2B may include example sensor(s) 216, such as a spatial sensor 218, an image sensor 220, and/or other sensors that may not be explicitly illustrated. The spatial sensor 218 may be and/or include one or more of a time of flight (ToF) depth sensor, an accelerometer, a gyroscope, a magnetometer, a GPS sensor, a radar sensor, or the like. In some implementations, one or more of the spatial sensor 218 may be integrated in a single inertial measurement unit.

Continuing with FIGS. 2A and/or 2B, in some embodiments, the image sensor 220 of any of the pretreatment computing device 104 or the augmented reality device 106 may be any combination of one or more infrared (IR) sensors, visible (or optical) light sensors, ultraviolet (UV) light sensors, X-ray sensors, and/or other image sensors used in, for example, a pretreatment process or an intraoperative process of a medical procedure. For example, the medical provider 120 of FIG. 1 may use the augmented reality device 106 during an intraoperative process of the medical procedure. In such a case, the spatial sensor 218 of the augmented reality device 106 may capture images (e.g., 2D images, digital images) of the real environment 116 of FIG. 1, track the position of the medical provider 120's head, track the position(s) of the medical provider 120's eye(s), iris(es), pupil(s), and/or so forth.

Examples of systems and methods described herein may implement and/or be used to implement techniques that, for example, the pretreatment computing device 104 of FIGS. 1 and/or 2A or other computing device(s) may utilize to convert 2D images into a 3D image and/or a 3D reconstructed model.

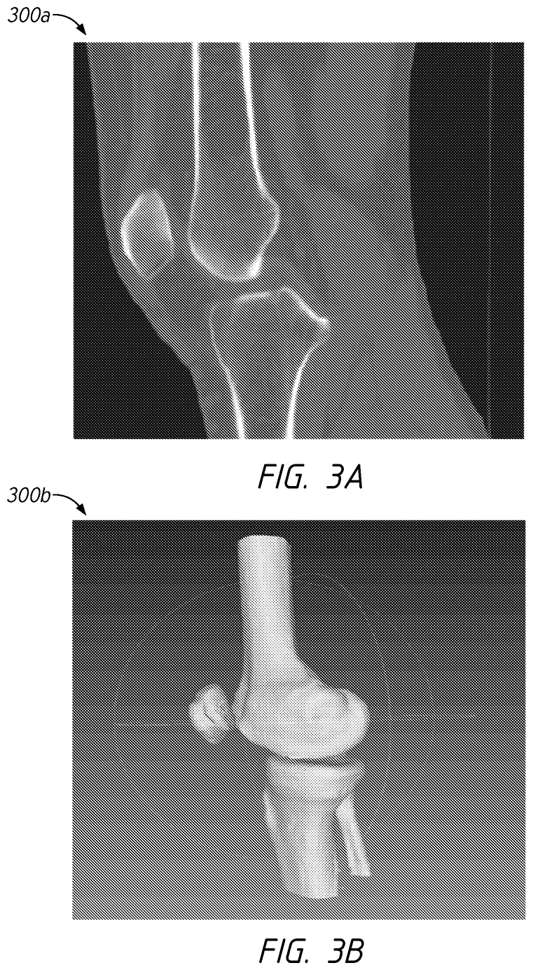

FIG. 3A illustrates an example 2D image 300a of a portion of a body in accordance with one embodiment. FIG. 3B illustrates an example 3D reconstructed model 300b of the portion of the body in accordance with one embodiment. In FIGS. 3A and 3B, the example portion of the body is a knee of the patient 118 of FIG. 1. Nevertheless, the techniques, methods, apparatuses, systems, and/or means described herein may be used during a medical procedure of other portions of the body including a hip, shoulder, foot, hand, or generally any anatomical feature(s). Furthermore, the techniques, methods, apparatuses, systems, and/or means described herein may be used for simpler (e.g., routine, outpatient) or more complex medical procedures that may have been explicitly described herein.

In one aspect, FIGS. 3A and 3B are partly described in the context of FIGS. 1 and 2A. For example, the instructions 214 of the pretreatment computing device 104 may cause the processor 210 of the pretreatment computing device 104 of FIG. 2A to execute a graphical reconstruction process. The graphical reconstruction process may convert 2D images (e.g., the 2D image 300a) into a 3D image and/or a 3D reconstructed model (e.g., the 3D reconstructed model 300b).

Examples of 2D images may be obtained using one or more medical imaging systems. Examples include magnetic resonance imaging (MRI) systems which may provide one or more MRI images, one or more computerized tomography (CT) systems which may provide one or more CT images, and one or more X-ray systems which may provide one or more X-ray images. Other systems may be used to generate 2D images in other examples, including one or more cameras.

In one aspect, the 2D images may be represented using a first file format, such as a digital imaging and communications in medicine (DICOM) file format. In another aspect, the 3D image and/or the 3D reconstructed model may be represented using a second file format, such as a point cloud, a 3D model, or the like file format.

In aspects, a conversion from a 2D image (e.g., a 2D DICOM image) to a 3D image (e.g., a 3D point cloud image, a 3D model) may be accomplished using a variety of techniques. For example, a volumetric pixel in a 3D space (e.g., a voxel) may be a function of the size of a 2D pixel, where the size of the 2D pixel may be a width along a first axis (e.g., x-axis) and a height along a second axis (e.g., y-axis). By considering the depth of the voxel along a third axis (e.g., a z-axis), the pretreatment computing device 104 may determine a location of a 2D image (or a 2D slice).

In some embodiments, in order to perform a conversion from the 2D images to a 3D image, the pretreatment computing device 104 may utilize DICOM tag values, such as, or to the effect of: i) a 2D input point (e.g., x, y): ii) an image patient position (e.g., 0020, 0032); iii) a pixel spacing (e.g., 0028, 0030): iv) a row vector and a column vector (e.g., 0020, 0037), and/or additional DICOM tag values.

To convert a 2D pixel to a 3D voxel, the instructions 214 of the pretreatment computing device 104 may include using the following equations (e.g., Equations 1, 2, and 3).

voxel(x,y,z)=(image plane position)+(row change in x)+(column change in y) Equation 1

row change in x=(row vector)·(pixel size in the x direction)·(2D pixel location in the x direction) Equation 2

column change in y=(column vector)·(pixel size in the y direction)·(2D pixel location in the y direction) Equation 3

Using the DICOM tag values (that may also be stored in the computer-readable medium 212 of the pretreatment computing device 104) and the Equations 1, 2, and 3, the pretreatment computing device 104 may convert the 2D image 300a of the knee of the patient 118 to the 3D reconstructed model 300b of the knee of the patients 118.

Additionally, or alternatively, the pretreatment computing device 104 and/or any device in the surgical navigation system 102 may utilize a variety of other techniques, equations, and/or software to convert a 2D DICOM file (e.g., the 2D image 300a) to various 3D files (e.g., the 3D reconstructed model 300b), including but not limited to: a 3DSlicer (open source, available at https://www.slicer.org/) and embodi3D (available at https://www.embodi3d.com/), which are both incorporated herein by reference in their entirety for any purpose. An example 3D file format may be a standard tessellation language (STL) that may be used in 3D printing. Another example 3D file format may be a TopoDOT® file. Therefore, different types of file formats for 3D reconstruction may be used for the 3D reconstructed model.

In some examples, a consistent file format may be used throughout the processing sequence, for example, in order to maintain integrity of the reconstructed pretreatment. A voxel with an x, y, and z coordinates may be identified by a coordinate of its center in a 3D space that may include the 3D reconstructed model (e.g., the 3D reconstructed model 300b). In the 3D reconstructed model, each voxel has a location that is referenced to each other, but not yet referenced to a location in actual and/or physical space. The ability to group voxels and isolate them from other voxels allows for the segmentation and identification of specific anatomical areas and structures.

Accordingly, the 3D reconstructed model may include data representing the model, and the data may be stored one or more computer readable media, including those described herein. The 3D reconstructed model may be displayed (e.g., visualized) using one or more display devices and/or augmented or virtual reality devices, including those described herein.

In some embodiments, the 3D reconstructed model (e.g., the 3D reconstructed model 300b) may be a basis for a surgical procedure planning, such as a pretreatment process of a medical procedure (e.g., a knee surgery). The 3D reconstructed model may be used for measurement(s) of a targeted anatomy, such as a section of interest of the portion of the body. For example, if the portion of the body is a knee, the section of interest may be a bone (e.g., a femur, a tibia, a patella), a cartilage (e.g., a medial meniscus, a lateral meniscus, an articular cartilage), a ligament (e.g., an anterior cruciate ligament (ACL), a posterior cruciate ligament (PCL), a medial collateral ligament (MCL), a lateral collateral ligament (LCL)), a tendon (e.g., a patellar tendon), a muscle (e.g., a hamstring, a quadricep), a joint capsule, a bursa, and/or a portion thereof (e.g., a medial condyle of the femur, a lateral condyle of the femur, and/or other portions may be the section of interest). The 3D reconstructed model may additionally or instead used for developing surgical plans (e.g., one or more resection planes, cutting guides, or other locations for surgical operations) relating to the targeted anatomy.

For example, in a total joint replacement procedure, implants replace the joint interface. Total joint replacement may be indicated due to wear or disease of the joint resulting in degradation of the bone interfaces. It may be beneficial to measure the size of the anatomy that is to be replaced (e.g., the section of interest of the portion of the body) in order to select the correct implant for use in the surgical procedure. The 3D reconstructed model (e.g., the 3D reconstructed model 300b) may be used to provide one or more measurements along any of the x, y, or z axes of the 3D reconstructed model, the section of interest, the portion of the body, or combinations thereof. In some implementations, the x, y, and z axes may be mutually orthogonal (e.g., a Cartesian coordinate system). In some implementations, other coordinate systems may be used, such as polar coordinates, cylindrical coordinates, curvilinear coordinates, or the like. The 3D reconstructed model can be used prior to surgery for planning. In some embodiments, the pretreatment computing device 104 may generate, provide, store, and/or display (collectively may be referred to as “provide”) the 3D reconstructed model. The pretreatment computing device 104 may execute instructions 214 during a pretreatment process by, for example, using an application program software that may reside in any computer-readable medium 212 of any of the devices of the surgical navigation system 102, or may reside on a server or a cloud that may not be explicitly illustrated in any of the figures of this disclosure.

In some embodiments, the pretreatment process may include: a medical provider (e.g., the medical provider 120 of FIG. 1 logging in and/or on the pretreatment computing device 104 of FIG. 2A: the medical provider inputting information of the patient (e.g., the patient 118 of FIG. 1): the medical provider selecting a medical procedure: the medical provider uploading 2D pretreatment DICOM images (e.g., the 2D image 300a of FIG. 3A) of a portion of a body (e.g., a knee) of the patient for the selected medical procedure: the pretreatment computing device 104 converting the 2D pretreatment DICOM images to a 3D reconstructed model (e.g., the 3D reconstructed model 300b of FIG. 3B): the pretreatment computing device 104 displaying, for example, on the display 204 of the pretreatment computing device 104, the 3D reconstructed model for pretreatment planning: and the medical provider identifying a targeted area (or a section of interest of the portion of the body, e.g., a medial condyle of the femur, a lateral condyle of the femur), and identifying, demonstrating, and/or displaying the section of interest on the 3D reconstructed model.

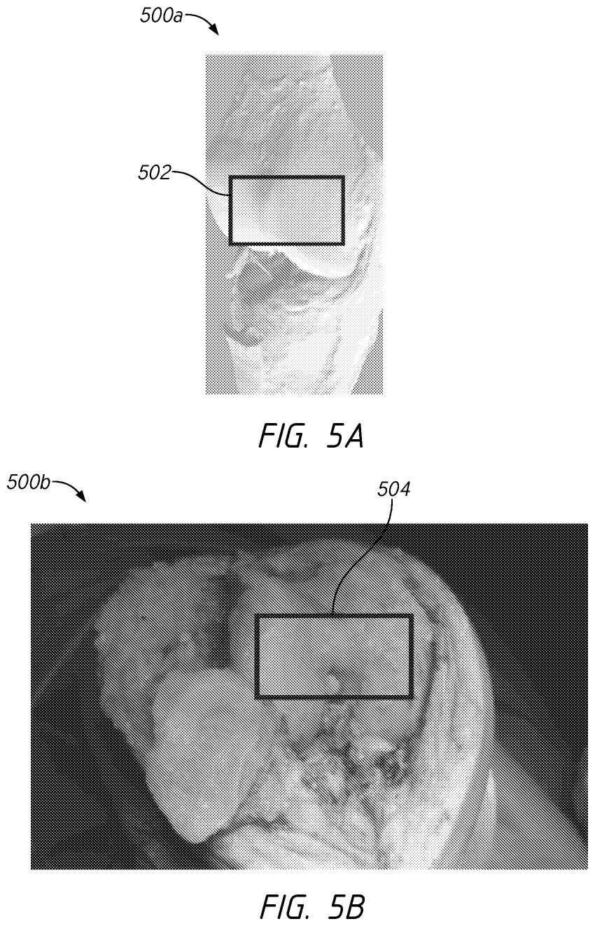

Accordingly, a 3D model (e.g., a 3D reconstructed model) of a patient's anatomy may be used to conduct pretreatment planning (e.g., to select resection planes, implant locations and/or sizes). A boundary may be defined around a location of interest in the 3D model, such as around the medial condyle of the femur and/or around the lateral condole of the femur, as is illustrated by a model boundary 502 in FIG. 5A. This model boundary may be used to register the 3D model to intraoperative images of the anatomy, as is illustrated by the intraoperative image 500b in FIG. 5B. The boundary may be two and/or three dimensional, and it may be sized to enclose a particular anatomical feature, such as the medial condyle of the femur and/or the lateral condyle of the femur. A number of pixels, voxels, and/or other data may be within the model boundary. It is these pixels, voxels, and/or other data which may be used for registration in examples described herein. The model boundary may be generally any size or shape and may be defined and/or drawn by the medical provider in some examples.

In aspects, the pretreatment computing device 104 may allow for preoperative planning on the virtual anatomy based on the patient's imaging prior to entering the operating suite. The pretreatment computing device 104 can also consider elements like the type of implant that may better fit the patient's anatomy. The implant type may be based on measurements taken from the 3D reconstructed model. Therefore, a preferred, an optimal, and/or a suitable implant for the patient may be determined based on the 3D reconstructed model (e.g., the 3D reconstructed model 300b). Other pretreatment or intraoperative planning models are contemplated within the scope of the present disclosure that incorporate the features of planning, placement and virtual fitting of implants, or virtual viewing of treatment outcomes prior to actual application in an intraoperative setting. The present disclosure provides techniques, methods, apparatuses, systems, and/or means to effectively translate pretreatment or intraoperative planning to the intraoperative setting in a practical manner. Examples of pretreatment processes and the intraoperative processes are described herein.

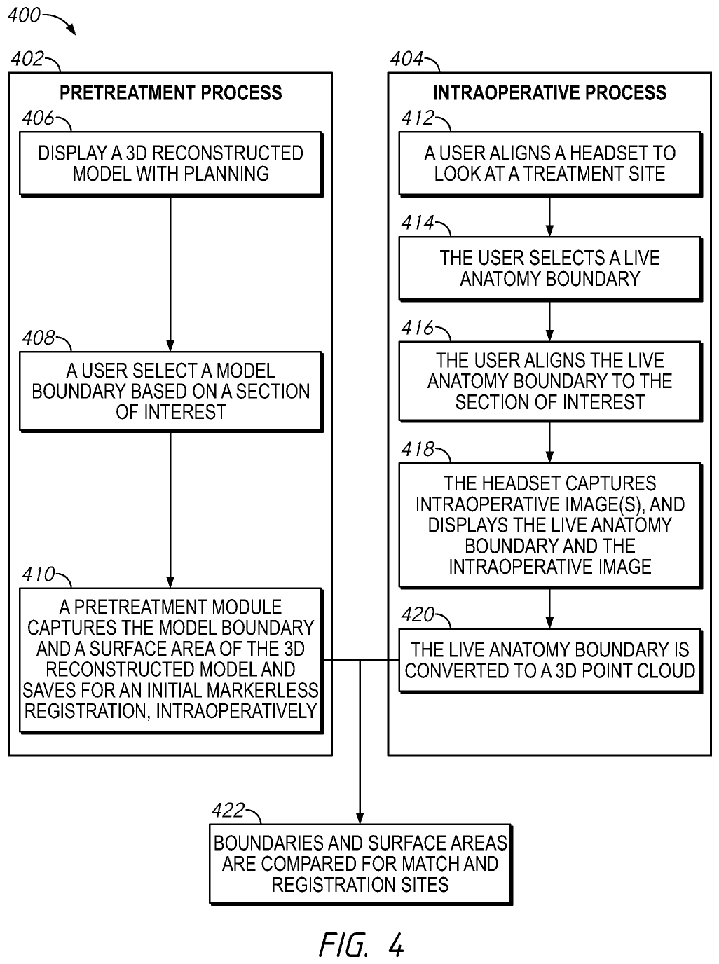

FIG. 4 illustrates an example method 400 for registering a 3D reconstructed model of a portion of a body of a patient with an intraoperative view of that portion of the body of the patient. As described herein, the 3D reconstructed model may be generated during a pretreatment process 402 of a medical procedure, and the intraoperative images of the actual portion of the body may be generated or captured during an intraoperative process 404 of the medical procedure. The pretreatment process may in some examples occur at a different time than the intraoperative process, such as before the intraoperative process. In some examples, the pretreatment process may occur minutes, hours, days, and/or weeks before the intraoperative process. The pretreatment process 402 may be implemented in some examples using a different computing system than the intraoperative process 404. For example, pretreatment computing device 104 of FIG. 1 may be used to perform all or portions of the pretreatment process 402. A model generated during that process may be stored (e.g., in memory) and/or communicated to another computing system, such as the registration computing device 110 of FIG. 1. The registration computing device 110 of FIG. 1 may perform all or portions of the intraoperative process 404 of FIG. 4.

According to the present disclosure, an example method to implement a pretreatment process (or intraoperative plan) may utilize augmented reality imaging to overlay a 3D reconstructed model of patient anatomy onto a view of an actual intraoperative treatment site. The pretreatment process 402 may be executed using the respective instructions 214 of the pretreatment computing device 104 of FIG. 1 in some examples. By so doing, the plan for treatment outlined in the 3D reconstructed model may become a guide for the surgical steps to achieve the planned and desired surgical outcome(s).

The augmented reality device 106 of FIG. 1 may be implemented using a mixed reality device that allows for both visualization of the actual treatment area and also generating an image (e.g., a virtual image and/or virtual object) that can be overlaid with the view of the actual treatment area. In many implementations, the overlaid image may be adapted to appear from the point of view of a wearer of an augmented reality device 106 to be present in the actual treatment area. In addition, the augmented reality device 106 can contain sensors (e.g., sensor(s) 216, the spatial sensor 218) for depth or location measurements and cameras (e.g., the image sensor 220) for visualization of the actual intraoperative anatomy.

In some example embodiments, methods (e.g., the example method 400) of the present disclosure may include identification of one or more specific sections of the patient's anatomy of interest (“section of interest”) of the 3D reconstructed model. The identification may be made, for example, by one or more users. For example, a medical provider, technician, or other human user may identify the section of interest using an interface to a pretreatment computing system, e.g., by drawing a boundary around the section of interest and/or moving a predetermined boundary shape onto the section of interest. For example, as is illustrated in FIG. 5A, the medical provider may draw a boundary (e.g., the model boundary 502) around the medial condyle of femur, the lateral condyle of the femur, both the medial and the lateral condyles, a portion of the medial condole, a portion of the lateral condyle, and/or another section of interest that may not be explicitly illustrated. In some examples, an automated process, e.g., a software process, which may be executed by the pretreatment computing system may position a boundary around a section of interest in an automated way. Accordingly, the section of interest of the 3D reconstructed model may be identified by a boundary, which may be referred to as a model boundary (e.g., the model boundary 502 of FIG. 5A). During intraoperative process 404, the same section of interest of the actual intraoperative site (e.g., live anatomy site, real environment 116 of FIG. 1) may also be identified by a boundary, which may be referred to as a live anatomy boundary, as is illustrated by a live model boundary 504 in FIG. 5B. During the intraoperative process 404, a medical provider or other human user may identify a location for the live anatomy boundary (e.g., by performing a gesture or other action recognizable to an augmented reality headset to generate and/or position a live anatomy boundary). In some examples, the live anatomy boundary may be selected and/or positioned using an automated process (e.g., software executed by the augmented reality headset and/or registration computing system and/or intraoperative computing system).

These boundaries (e.g., the model boundary, the model boundary 502, the live anatomy boundary, the live model boundary 504) may be represented with a myriad variety of shapes and forms. For example, a boundary may be a two-dimensional area. The two-dimensional area may be defined by one or more of geometric shapes, and the one or more geometric shapes may include a line, a regular polygon, an irregular polygon, a circle, a partial circle, an ellipse, a parabola, a hyperbola, a logarithmic-function curve, an exponential-function curve, a convex curve, a polynomial-function curve, or a combination thereof. As another example, a boundary may be planar, or may be a surface with relief (e.g., an area that while two dimensional includes information about topology of the patient's anatomy similar to a topographic map). As yet another example, a boundary may be a three-dimensional volumetric region. The three-dimensional volumetric region may be defined by a cuboid, a polyhedron, a cylinder, a sphere, a cone, a pyramid, a prism, a torus, or combination thereof, and/or any other three-dimensional volumetric region. A boundary may accordingly generally have any shape and form, including a shape and a form that may be selected and/or drawn (e.g., manually drawn) by a medical provider or a medical professional.

In many examples, the one or more identified sections of interest may be associated with (e.g., represented by) one or more boundaries, for example, rectangles and/or bounding boxes, as is illustrated by a model boundary 502 of a 3D reconstructed model 500a in FIG. 5A and a live anatomy boundary 504 of an intraoperative image 500b of FIG. 5B. A boundary (e.g., a bounding box) may be positioned by a user, which may be another software process positioning the boundary in an automated way, to define digital sampling on a 3D reconstructed model. The boundary is generally positioned to enclose an anatomical feature of interest (e.g., the medial condyle of femur, the lateral condyle of the femur). In some implementations, the section of interest represented by the boundary may be predetermined by a user (e.g., a medical provider, a first medical provider, the medical provider 120, and/or another automated software process which may store one or more sections of interest in memory). In some implementations, a user (e.g., a medical provider, a second medical provider, the medical provider 120) may manually place a bounding box on the live intraoperative anatomy of the patient to approximate the position of a section of interest of a portion of a body. For example, the medical provider 120 may perform a gesture or utilize an input device (e.g., mouse, trackpad) to position a virtual image of a boundary as viewed through the headset superimposed on the intraoperative view of the anatomy. In some examples, the medical provider 120 may utilize an input device (e.g., mouse, trackpad) to position a boundary on an image of the intraoperative anatomy as displayed by a computing device (e.g., an image taken from one or more cameras during an intraoperative process). In some implementations a system, an apparatus, an application software, modules of the application software, an algorithm, a model, and/or a combination thereof that may be disclosed herein can estimate the position of the section of interest based on edge detection of the live exposed anatomy and the relationship of the edges of the boundary to the 3D reconstructed model. While examples are described herein with reference to bounding box(es), it is to be understood that other shaped boundaries may be used in other examples.

In some implementations, a boundary may be created from a digital image captured intraoperatively, such as by the augmented reality device 106 (e.g., a live anatomy boundary, the live anatomy boundary 504 of FIG. 5B). In some implementations, a boundary may be created in the 3D reconstructed model (e.g., a model boundary, the model boundary 502 of FIG. 5A). For example, one or more model boundaries may be created in the 3D reconstructed model, for example, during a pretreatment process 402. The surface of a model boundary may have an identifiable pattern, shape, and/or form. The identifiable pattern, shape, and/or form may be utilized for comparison to a live anatomy boundary, such as for use in a registration process whereby the 3D reconstructed model may be aligned with the patient's actual anatomy.

In some examples, the size and/or shape of the boundary positioned during the intraoperative process may be based on the size and/or shape of the boundary positioned during the pretreatment process. For example, a computing device (e.g., augmented reality headset) may size a boundary to match a size of a boundary used during pretreatment. For example, if a pretreatment boundary was drawn on a model of the anatomy, a pretreatment computing device may measure a size of the pretreatment boundary and/or obtain measurements of the pretreatment anatomy. For example, based on a scale of the pretreatment model, a boundary may be determined to present a 2 cm×2 cm×2 cm section of 3D anatomy, and/or a 2 cm×2 cm section of 2D anatomy. Other sizes may be used in other examples. During an intraoperative process, an augmented reality headset may size a boundary based on a view of the anatomy and position of the headset to provide a same sized boundary for overlay on the intraoperative anatomy—e.g., a boundary that encloses a 2 cm×2 cm section of intraoperative anatomy.

Examples described herein may utilize the boundaries placed on model anatomy (e.g., 3D pretreatment model and/or 2D pretreatment images) and intraoperative anatomy to register the model with intraoperative anatomy. In some embodiments, the registration process may be executed by the registration computing device 110 of FIGS. 1 and/or 2D, such as by using the instructions 214 of the registration computing device 110 of FIG. 2D. This approach may include some advantages: for example, this approach may not randomly match all images of the actual treatment site to the 3D reconstructed model. Moreover, the registration computing device 110 may not match the entire intraoperative image 500b of FIG. 5B to the entire 3D reconstructed model 500a of FIG. 5A. Such an approach may in some examples better perform in the presence of noise during image processing and analysis on both sides of the image capturing and reconstruction. Matching of selected digital data (e.g., digital samples such as pixels and or voxels) from the area of the reconstructed model and/or pretreatment image within the boundary may be performed to the digital data (e.g., pixels) from within the boundary placed on an intraoperative image. This matching may provide an orientation and direction that may be used (e.g., by registration computing systems and/or augmented reality headsets and/or surgical navigation systems described herein) to overlay areas of the 3D model which are outside of the boundary onto the intraoperative scene. Accordingly, the entire 3D model may be registered to and/or displayed overlaid on the intraoperative scene utilizing data from a boundary region of interest. For example, additional portions of the 3D model other than just the boundary area may be registered to the intraoperative image based on a matching that is performed using only the boundary area (or volume). The boundary area (or volume) may make up a fraction of an area (or volume) of the entire model. In some examples, the boundary area may be 10% or less of the entire model area registered to a preoperative image. In some examples, the boundary area may be 5% or less of the entire model area registered to a preoperative image. In some examples, the boundary area may be 20% or less of the entire model area registered to a preoperative image. Other percentages may be used in other examples.

Note that noise may be a significant problem in a medical practice when trying to use image analysis alone for navigation systems. Noise may also lead to an error in the 3D reconstruction and/or an error in the positioning (e.g., registration) of the reconstruction to the actual patient anatomy. Noise may include unwanted data received with or embedded in a desired signal. For example, noise may include random data included in a 2D image, such as detected by an x-ray detector in a CT machine. Noise may also be and/or include unwanted data captured by a camera (e.g., the image sensor 220) of a head-mounted display (HMD) (e.g., the augmented reality device 106). Noise of the camera of the HMD may be due to the camera being pushed to the limits of its exposure latitude: consequently, a resulting image can have noise that may show up in the pixels of the image. Noise may also be related to an anatomical feature not associated with the current surgical procedure being planned. By using a digital sample from one or more specific boundaries within the 3D reconstructed model, extraneous noise may be reduced and/or removed due to the selection of a known reconstructed area through the narrowing of the digital sample selected. For example, attempting to register the 3D reconstructed model with an image of an actual patient anatomy may be prone to error due to significant portions of the patient anatomy corresponding to the model contributing noise to the registration process.

Utilizing only particular areas, identified by boundaries (e.g., the model boundary 502, the live anatomy boundary 504, which may be positioned around the medial and/or lateral condyle of the femur), to register the 3D reconstructed model with the actual patient anatomy may allow for registration that is more resilient to noise or error in the model and/or intraoperative images. In some implementations, a boundary may be used as a same-size comparator that can be positioned virtually in a near location on the actual anatomy. The same-size comparator may include a limited window of the intraoperative surgical field against which the 3D reconstructed model is matched in the registration process. In some implementations, the same-size comparator may include all or most of the data used to match the intraoperative environment (e.g., the intraoperative image 500b, the real environment 116) to the 3D reconstructed model (e.g., the 3D reconstructed model 500a).

In FIG. 4, a user during the pretreatment process 402 may be the same as, or different from, a user during the intraoperative process 404. For example, a first medical provider (e.g., a CT scanner technician, a first medical doctor) may be involved during the pretreatment process 402, and a second medical provider (e.g., a surgeon, a second medical doctor) may be involved during the intraoperative process 404. As another example, the same medical provider (e.g., a surgeon) may be involved during the pretreatment process 402 and the intraoperative process 404. Therefore, the term “user” in FIG. 4 may denote all these scenarios, and/or other scenarios, including use of one or more automated software processes to select and/or position boundaries described herein.

In addition, blocks of the example method 400 (or of any other method described herein) do not necessarily need to be executed in any specific order, or even sequentially, nor need the operations be executed only once. Furthermore, the example method 400 can be utilized by using one, more than one, and/or all the blocks that are illustrated in FIG. 4. Therefore, the example method 400 does not necessarily include a minimum, an optimal, or a maximum number of blocks that are needed to implement the systems, methods, and techniques described herein.

The pretreatment process 402 may be executed by the pretreatment computing device 104, such as by the processor 210 executing the instructions 214 of the computer-readable medium 212 of the pretreatment computing device 104 of FIG. 2A. If the pretreatment computing device 104 of FIG. 2A includes a display 204, in some embodiments, at block 406, the pretreatment process 402 may include displaying the 3D reconstructed model (e.g., the 3D reconstructed model 300b, the 3D reconstructed model 500a) on the display 204 of the pretreatment computing device 104. By so doing, the medical provider and/or the user can at least see, observe, study, and/or reference the 3D reconstructed model, and the medical provider and/or the user may create a treatment plan for the medical procedure.

At block 408 of the pretreatment process 402, the user may select a model boundary (e.g., the model boundary 502 of FIG. 5A). The model boundary selection may be done in a variety of ways. For example, the user may manually position the model boundary based on a section of interest of a portion of a body of the patient. In a manual approach, the user may be prompted to look at a comparative area on the patient's anatomy that matches the location of the model boundary in the 3D reconstructed model. In this approach, the 3D reconstructed model (e.g., the 3D reconstructed model 500a of FIG. 5A) and the model boundary (e.g., model boundary 502) can be displayed, for example, on the display 204 of the pretreatment computing device 104. Alternately, just the model boundary may be displayed on the display 204. Either or both the 3D reconstructed model and/or the model boundary may be overlaid on a view of the patient's anatomy, such as through the augmented reality device 106.

In some examples, a user may be prompted to position the boundary such that it contains all or a portion of a particular anatomical feature. The particular anatomical feature may be one which contains detail that is advantageous to matching to a subsequent intraoperative image. For example, a feature having variability and/or likely to have a lesser amount of noise than the total image and/or model.

In some implementations, the model boundary can be positioned using a boundary positioning technique that analyzes one or more images of the intraoperative treatment area using techniques such as machine-learned algorithms, image classifiers, neural network processes, edge detection, and/or anatomy recognition. A boundary positioning technique may probabilistically determine the likely location in the patient's actual anatomy of the comparative location in the 3D reconstructed model. The model boundary can be a virtual boundary created in the 3D reconstructed model such as by manual drawing, so the boundary has a specific size, shape, form, and/or location in the 3D reconstructed model. A corresponding live anatomy boundary may be created with a same (or approximately the same) size, shape, and form as the model boundary. In some examples, the live anatomy boundary may be a different size, shape, and/or form has the model boundary. The user may place the live anatomy boundary on (or overlay over) a view of the actual treatment site. The live anatomy boundary can be a virtual boundary that takes a digital sample of a specific size, shape, form, and location on the pretreatment model (e.g., the 3D reconstructed model) and a corresponding digital sample that is the same size, shape, and form to be placed on or overlaid over the actual treatment site. One or more of each of the model and live anatomy boundaries can be used as desired. Using multiple boundaries may increase fidelity and/or speed of registration between the 3D reconstructed model and the patient's anatomy (e.g., portion of the body of the patient). The boundary (e.g., the live anatomy boundary) can be placed automatically as a virtual overlay of the actual treatment site, for example, based on the image analysis of a live video feed of the actual treatment site. The boundary can be placed automatically as an overlay of the patient's anatomy on the actual treatment site in some examples based on the surface mapping of the actual treatment site. While in some examples the live anatomy boundary may be placed as a virtual object, in some examples, the live anatomy boundary may be positioned on an image of the anatomy taken during an intraoperative procedure.

In some embodiments, at block 410, the pretreatment computing device 104 may utilize a pretreatment module (e.g., a portion of an application software) that may be stored and/or accessed by the computer-readable medium 212 of the pretreatment computing device 104. The pretreatment module may capture the model boundary and a surface area of the 3D reconstructed model. The pretreatment module may also save the model boundary and the surface area of the 3D reconstructed model for an initial markerless registration, for example, for later use, such as during the intraoperative process 404.

The intraoperative process 404 may be partly or wholly executed by the augmented reality device 106, such as by the processor 210 executing the instructions 214 of the computer-readable medium 212 of the augmented reality device 106 of FIG. 2A. Alternatively, some blocks of the intraoperative process 404 may be executed by another computing device of the surgical navigation system 102, such as the registration computing device 110 and/or the surgical navigation computing device 108.

In some embodiments, at block 412 of the intraoperative process 404, a user (e.g., a surgeon, a medical provider 120) may align a headset (e.g., the augmented reality device 106) to look at a treatment site. The treatment site may be a portion of a body of a patient (e.g., the patient 118). The display of the headset (e.g., the display 204 of the augmented reality device 106) may display an intraoperative image and at least one live anatomy boundary based on the intraoperative image. The user may position the headset such that the portion of the body, including the desired anatomical feature for association with a live anatomy boundary, is visible when viewed from the headset.

In some embodiments, at block 414, the headset (e.g., the augmented reality device 106) may automatically select a live anatomy boundary having the same size, shape, and/or form as the model boundary created on block 408 of the pretreatment process 402. In some embodiments, the headset may display more than one live anatomy boundary for the user to choose from. In short, the headset aids the user to select and/or position the live anatomy boundary (e.g., the live anatomy boundary 504).

After the selection of the live anatomy boundary, at block 416 of the intraoperative process 404, the user aligns the live anatomy boundary with the section of interest of the portion of the body of the patient (e.g., the patient 118). After the alignment of the live anatomy boundary (e.g., the live anatomy boundary 504) with the section of interest, at block 418, the headset may capture intraoperative image(s) and displays (e.g., on a display 204 of the augmented reality device 106) the live anatomy boundary and the captured intraoperative image.

In some embodiments, at block 420 of the intraoperative process 404, the augmented reality device 106 and/or any other computing device in the surgical navigation system 102 may convert the live anatomy boundary to a 3D point cloud. For example, the pixels, voxels, and/or other data representative of the anatomy contained within the live anatomy boundary may be converted to a point cloud representation. In other examples, other data manipulations may be performed on the data within the live anatomy boundary including compression, edge detection, feature extraction, and/or other operations. One or more intraoperative computing device(s) and/or augmented reality headsets may perform such operations.

In some embodiments, at block 422, the boundaries (e.g., the model boundary and the live model boundary) and the surface areas (e.g., a surface area of the 3D reconstructed model and a surface area of the intraoperative image) are compared for matching and/or registration sites. Matching may be performed by rotating and/or positioning the data from within the model boundary to match the data from within the live anatomy boundary. In some examples, features may be extracted from the data within the model boundary and within the live anatomy boundary, and an orientation and/or position shift for the model to align the model with the live anatomy may be determined, e.g., using one or more registration computing devices or another computing device described herein. Using the orientation and/or position shift to align the features within the boundary areas of the model and the live anatomy, additional portions of the model other than the boundary area (e.g., the entire model) may be accordingly depicted, superimposed, or otherwise aligned to the live anatomy. Note that the alignment of the entire model is based on an analysis (e.g., matching) of data within one or more boundary areas. Because the entire model and/or entire live anatomy view is not used in the registration or matching process in some examples, the registration process may be more tolerant to noise or other irregularities in the model and/or intraoperative image.

If the comparison includes less than a predetermined error threshold (e.g., a difference threshold), the user utilizes the matched boundaries and surface areas to perform the medical procedure. If, however, at block 422, the boundaries and the surface areas do not match, the processes described in some of the blocks of the example method 400 (e.g., blocks 410, 412, 416, 418, 420, and/or 422) may be repeated until the comparison includes less than the predetermined error threshold. Therefore, in some embodiments, the example method 400 may be an iterative process.

FIG. 5A illustrates an example model boundary 502 of an example 3D reconstructed model 500a that may be generated during a pretreatment process of the medical procedure; and FIG. 5B illustrates a corresponding live anatomy boundary 504 of an example intraoperative image 500b that may be generated during an intraoperative process of the medical procedure.

In some embodiments, boundaries (e.g., model boundaries, live anatomy boundaries) can be used strategically based on the type of procedure to identify likely exposed anatomy. This may be particularly useful when, for example, the exposed anatomy is minimally visible due to a less invasive surgical approach. The registration of the 3D reconstructed model to minimally visible surgical sites make it possible to use the restricted view in a more meaningful way. For example, the location of the surgical incision alone in a boundary, may reveal location information in relationship to the entire surgical anatomy that can be used to approximate the initial placement of the 3D reconstructed model. Inside the surgical incision, any exposed surgical anatomy can be used for comparison to the 3D reconstructed model for matching and registration.

The boundaries can be used in conjunction with sensors (e.g., sensor(s) 216) like depth cameras with active infrared illumination, for example, mounted to or otherwise included in an augmented reality device 106 for spatial mapping of the surgical site. The depth measurements along with other sensors, like accelerometers, gyroscopes, and magnetometers, may provide real-time location information that may be useful for real-time tracking of the movement of the augmented reality device 106. Thus, the location of the boundary may be generated live in relationship to the actual surgical site. Other mechanisms, like a simultaneous localization and mapping (SLAM) algorithm from live video feeds of the surgical site can be used for spatial relationship between the scene of the video and the augmented reality device 106. The scene may contain the boundary (e.g., the live anatomy boundary), and thus the boundary may have a spatial relationship within the scene and referenceable to the augmented reality device 106. This process may allow for continual updating of the initial tracking between the 3D reconstructed model and the patient's anatomy, for example, to account for movement of the boundary (e.g., live anatomy boundary) within the scene.

Matching data within the two or more boundaries (e.g., the model boundary 502 of FIG. 5A and the live anatomy boundary 504 of FIG. 5B) can be done in a variety of different ways. For example, for respective instructions 214 of the augmented reality device 106 and/or registration computing device 110 may include and/or utilize an iterative closest point (ICP) algorithm. In an ICP or, in some implementations, an ICP algorithm, one point cloud (e.g., a vertex cloud representing the reference, or target), is kept fixed, while another point cloud (e.g., a source), is transformed to best match the reference. As another example, patterns within each boundary can be detected and then compared to each other using, for example, a machine-learned model. As yet another example, the respective instructions 214 of the augmented reality device 106 and/or the registration computing device 110 may utilize the ICP algorithm in combination with the machine-learned model to better match digital samples from within the model boundary 502 with digital samples from within the live anatomy boundary 504 to register the 3D reconstructed model 500a with the intraoperative image 500b and/or the portion of the body of the patient. Matching the digital samples from within the model boundary with the digital samples from within the live anatomy boundary may reduce and/or obviate a need for a fiducial, a tracker, an optical code, a tag, or a combination thereof to perform registration.

Boundaries can also be utilized in conjunction with edge detection as a method of initially registering the 3D reconstructed model to the live anatomy. In this approach, edge detection employs the use of mathematical models to identify the sharp changes in image brightness that are associated with the edges of an object. When applied to a digitized image of the live anatomy and a 3D reconstructed model, the edges of each object can be considered a boundary. The shape and location of the boundary may be the edges of the targeted anatomy (or section of interest portion of the body). The digital sample contained in each boundary created by the edge detection of the model and the live anatomy may be used for ICP or other types of matching at a more detailed level to ensure precision of the registration.

For example, the model boundary 502 of FIG. 5A and the live model boundary 504 of FIG. 5B may include a considerable portion of the medial condyle of the femur. It is to be appreciated that during preparation for a knee surgery (e.g., the pretreatment process 402) and during the knee surgery (e.g., the intraoperative process 404), the medial and/or condyle of the femur includes a unique and/or a distinctive pattern(s), shape, texture(s), size, and/or other unique and/or distinctive features compared to other portions of the knee. These unique and/or distinctive features may increase the precision of the registration when the portion of the model and intraoperative image containing the lateral and/or medial condyle is used to register the intraoperative image with the model. Accordingly, model boundaries and live boundaries described herein may generally be positioned about features which may be advantageously used for registering the model to the intraoperative image.fw

In another embodiment, the boundaries are used in conjunction with light detection and ranging (which may be referred to as LIDAR, Lidar, or LiDAR) for surface measurements preoperatively and intraoperatively to register the pretreatment model (e.g., the 3D reconstructed model, the 3D reconstructed model 300b, the 3D reconstructed models 500a). In this embodiment, a LiDAR scanner creates a 3D representation of the surface of the anatomy pretreatment at or near the targeted anatomy, particularly in the case of minimally invasive procedures that have limited visual field of the actual surgical target. The LiDAR-scanned area can employ boundaries to limit the digital samplings of each area to reduce noise, create targeted samples, allow for specific types of samples all in an effort to increase the probability of matching the model to the live anatomical site, without the need of, or a reduced count of, markers, trackers, optical codes, fiducials, tags, or other physical approaches used in traditional surgical navigation approaches to determine the location.

Surgical navigation systems described herein, such as the surgical navigation system 102, can be utilized in a variety of different surgical applications of devices, resection planes, targeted therapies, instrument or implant placement or complex procedural approaches. In one example, the surgical navigation system 102 can be used for total joint applications to plan, register, and navigate the placement of a total joint implant. The pretreatment image of the patient may be converted from a DICOM output to a 3D reconstructed model. The 3D reconstructed model may be used to measure and plan the optimal (e.g., better, more accurate) position of the joint implant. The measurements can include those needed to determine correct sizing, balancing, axial alignment, dynamic adjustments, placement of resection guides and/or placement of robotic arm locations for implant guidance. The 3D reconstructed model may include at least one model boundary (e.g., the model boundary 502) that is used in concert with a corresponding live anatomy boundary (e.g., live anatomy boundary 504) attained in live imaging (e.g., the intraoperative image 500b) of the targeted surgical anatomy. The live image can be obtained from an augmented reality (e.g., mixed reality) 106 or another camera and sensor device used to image and process the images obtained. The digital sampling from the live anatomy boundary may be compared, and the digital samples are processed for matching with digital sampling from along and/or within a model boundary. Once the digital samples of the live anatomy boundary are matched, the model may be virtually overlaid on the live anatomy in a pre-registration mode. The live anatomy may optionally be sampled again, with the same boundary and/or a different sampling. The new samples may be matched using a technique like ICP and/or other image processing techniques to match the 3D reconstructed model and the live anatomy in a more precise manner. In some instances, a second sample is not needed, and the original sample can be processed for ICP matching and registration. Once the images are aligned at the voxel level, the full 3D reconstructed model can be used to locate or inform the planning, placement, resection, and or alignment of the joint implant. The joint implant can be a knee implant, hip implant, shoulder implant, spine implant, or ankle implant. Other implants or devices may be placed, removed, and/or adjusted in accordance with markerless navigation techniques described herein in other examples.

In another example, the surgical navigation system 102 is used for repair of anatomical sites related to injury to plan, register, and navigate the repair of the site. The pretreatment image of the patient may be converted from a DICOM output to a 3D reconstructed model, as is described in FIGS. 3A and 3B. The 3D reconstructed model (e.g., the 3D reconstructed model 300b, the 3D reconstructed model 500a) may be used to measure and plan the optimal position of the repair. The measurements can include those needed to determine correct sizing, balancing, axial alignment, dynamic adjustments, placement of resection guides or placement of robotic arm locations for repair guidance. The 3D reconstructed model may include at least one boundary (or model boundary) used in concert with a corresponding live anatomy boundary attained in live imaging of the targeted surgical anatomy. The live image can be obtained from an augmented reality (e.g., mixed reality) device 106 or another camera and sensor device used to image and process the images obtained. The digital sampling from the live anatomy boundary may be compared and the digital samples processed for matching with a model boundary. Once the digital samples of the boundary (ies) are matched, the 3D reconstructed model may be virtually overlaid on the live anatomy in a preregistration mode. The live anatomy may optionally be sampled again, with the same boundary and/or a different sampling. The new samples may be matched using a technique like ICP and/or other image processing techniques to match the 3D reconstructed model and the live anatomy (e.g., the portion of the body of the patient) in a more precise manner. In some instances, a second sample is not needed, and the original sample can be processed for ICP matching and registration. Once the images are aligned at the voxel level, the model can be used to locate or inform the planning, placement, resection, and or alignment of the surgical repair plan. The repair can include optimal placement of anchors used during an ACL repair, an MCL repair, a UCL repair, or other surgical sites that require precise placement of anchoring devices as part of the repair process. These types of procedures are commonly known as Sports Medicine Procedures, and the repairs can be part of restoring a patient function so they can perform at a level equivalent to or better than prior to injury. As such, precise navigation of the surgical site is needed to ensure a high degree of accuracy in the repair process.

Generally, once a model, which may include a pretreatment plan, is registered to live anatomy described herein, one or more surgical navigation systems (e.g., surgical navigation computing device) may be used to aid in a surgical procedure in accordance with the pretreatment plan. For example, cutting guides, resection planes, or other surgical techniques may be guided using surgical guidance based on the pretreatment plan, now registered to the live anatomy.

The particulars shown herein are by way of example and for purposes of illustrative discussion of the preferred embodiments of the present invention only and are presented in the cause of providing what is believed to be the most useful an readily understood description of the principles and conceptual aspects of various embodiments of the invention in this regard, no attempt is made to show structural details of the invention in more detail than is necessary for the fundamental understanding of the invention, the description taken with the drawings and/or examples making apparent to those skilled in the art how the several forms of the invention may be embodied in practice.

The description of embodiments of the disclosure is not intended to be exhaustive or to limit the disclosure to the precise form disclosed. While the specific embodiments of, and examples for, the disclosure are described herein for illustrative purposes, various equivalent modifications are possible within the scope of the disclosure, as those skilled in the relevant art will recognize.

Specific elements of any foregoing embodiments can be combined or substituted for elements in other embodiments. Moreover, the inclusion of specific elements in at least some of these embodiments may be optional, wherein further embodiments may include one or more embodiments that specifically exclude one or more of these specific elements. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

What is claimed is:

1. A surgical navigation method comprising:
receiving a plurality of two-dimensional images of at least a portion of a body of a patient;
generating, from the plurality of two-dimensional images, a three-dimensional reconstructed model of the at least the portion of the body;
generating a model boundary in the three-dimensional reconstructed model based on a section of interest;
receiving an intraoperative image of the at least the portion of the body;
generating a live anatomy boundary based on the intraoperative image; and
matching digital samples from within the model boundary with digital samples from within the live anatomy boundary to register the three-dimensional reconstructed model with the at least the portion of the body.

2. An at least one non-transitory computer-readable storage medium including instructions that, when executed by at least one processor, configure the at least one processor to perform the method of claim 1.

3. The surgical navigation method of claim 1, wherein said matching of the digital samples from within the model boundary with the digital samples from within the live anatomy boundary obviates a need for a fiducial, a tracker, an optical code, a tag, or a combination thereof.

4. The surgical navigation method of claim 1, wherein said receiving of the intraoperative image comprises obtaining the intraoperative image using an augmented reality device during a medical procedure.

5. The surgical navigation method of claim 1, wherein the model boundary aids a medical provider during a pretreatment process, a preoperative process, an intraoperative process, a postoperative process, or a combination thereof of a medical procedure.

6. The surgical navigation method of claim 1, wherein the matching of the digital samples from within the model boundary with the digital samples from within the live anatomy boundary is performed by utilizing:
an iterative closest point algorithm;
a machine-learned model for matching one or more patterns of the digital samples from within the model boundary to one or more patterns of the digital samples from within the live anatomy boundary: or
a combination thereof.

7. The surgical navigation method of claim 1, wherein the model boundary comprises a two-dimensional area, the two-dimensional area being defined by one or more of geometric shapes, and the one or more geometric shapes comprising a line, a regular polygon, an irregular polygon, a circle, a partial circle, an ellipse, a parabola, a hyperbola, a logarithmic-function curve, an exponential-function curve, a convex curve, a polynomial-function curve, or a combination thereof.

8. The surgical navigation method of claim 1, wherein the model boundary comprises a three-dimensional volumetric region, the three-dimensional volumetric region being defined by a cuboid, a polyhedron, a cylinder, a sphere, a cone, a pyramid, a prism, a torus, or a combination thereof.

9. The surgical navigation method of claim 1, wherein the model boundary comprises a surface with a relief.

10. The surgical navigation method of claim 1, wherein the model boundary comprises a shape, the shape being drawn by a medical professional.

11. The surgical navigation method of claim 1, wherein the live anatomy boundary comprises approximately a same size, shape, form, location on the portion of the body, or a combination thereof as the model boundary.

12. A system for aiding a medical provider during a medical procedure, the system comprising:
an augmented reality headset;
at least one processor; and
at least one non-transitory computer-readable storage medium including instructions that, when executed by the at least one processor, cause the system to:
receive an indication of a live anatomy boundary for an intraoperative scene;
display, using the augmented reality headset, the live anatomy boundary overlaid on the intraoperative scene;
receive an indication of an alignment of the live anatomy boundary with a section of interest of at least a portion of a body; and
match a section of a pretreatment image defined by a pretreatment boundary with a section of an intraoperative image associated with the live anatomy boundary to register the pretreatment image with the intraoperative scene.

13. The system of claim 12, wherein the instructions, when executed by the at least one processor, further cause the system to match digital samples from within the live anatomy boundary with digital samples from within a model boundary associated with the pretreatment image of the portion of the body.

14. The system of claim 13, wherein the model boundary is based on a three-dimensional reconstructed model of the portion of the body.

15. The system of claim 14, wherein the matching of the digital samples aid the system to register the three-dimensional reconstructed model with the at least the portion of the body.

16. The system of claim 12, wherein the system comprises a markerless surgical navigation system.

17. The system of claim 12, wherein the instructions, when executed by the at least one processor, further cause the system to establish communication between the augmented reality headset and one or more of a pretreatment computing device, a surgical navigation computing device, and a registration computing device.

18. The system of claim 17, wherein the live anatomy boundary comprises a virtual object.

19. The system of claim 17, wherein the instructions, when executed by the at least one processor, further cause the system to:
generate a model boundary from a first input of a first medical professional during a pretreatment process of the medical procedure, the first input comprises the first medical professional utilizing the pretreatment computing device; and
generate the live anatomy boundary from a second input of a second medical professional during an intraoperative process of the medical procedure, the second input comprises the second medical professional utilizing the augmented reality device to: indicate the live anatomy boundary of the intraoperative image; indicate the alignment of the live anatomy boundary with the section of interest of the at least a portion of the body; or a combination thereof.

20. The system of claim 17, wherein the instructions further cause the system to provide guidance for a surgical procedure based on a registration of the pretreatment image with the intraoperative scene.
